(19) United States
(12) Patent Application Publication
(10) Pub. No.: US 2025/0169716 A1
(43) Pub. Date: May 29, 2025

(54) SYSTEM AND METHOD FOR USE IN PATIENT TRACKER MOVEMENT DETECTION

(71) Applicant: BIOSENSE WEBSTER (ISRAEL) LTD., Yokneam (IL)

(72) Inventors: SHAKED BAHARAV, TEL AVIV (IL); URIEL HOD, EIN AYALA (IL); IRIS SEGAL, QIRYAT MOTZKIN (IL); MICHAL BEN SHLOUSH, JADERA (IL)

(21) Appl. No.: 18/519,388

(22) Filed: Nov. 27, 2023

(51) Int. Cl. A61B 5/11; A61B 5/00

(52) U.S. Cl. CPC A61B 5/1114; A61B 5/746

(57) ABSTRACT

A method and system are presented, the method comprise: providing location data from one or more patient trackers (PT) placed on a patient's body; processing said location data and identifying translation data indicative of translation movement of the PT and rotation data indicative of angular orientation variation of the PT; determining a correlation between said translation data and said rotation data; and generating output signal (i) indicative of PT movement if correlation exceeds a selected threshold, or (ii) comprising an alert on PT movement with respect to patient if correlation is below said selected threshold. The method and system thereby enable differentiating between PT movement with patient's body part and PT movement with respect to patient body part.

### TECHNOLOGICAL FIELD

The present disclosure relates to techniques for assessing patient's head location and specifically relates to detection of movement of patient tracker with respect to the patient.

### BACKGROUND

Patient Tracker (PT) is a small element including one or more location or movement detectors that is configured to be placed on a patient's body. PTs are often used to compensate for head movement during selected procedure, e.g., medical procedures. Typically, a PT is placed on the forehead of a patient and transmits data on patient's movement. The PT movement data enables to compensate for the location of navigated tools as the patient's head moves.

U.S. Pat. No. 11,123,144 Describes an apparatus, including a patient tracker, attached to a subject, having magnetic field sensors and optical landmarks with known spatial relationships to each other. A camera acquires a 3D optical image, in a first frame of reference (FOR), of the subject's face. A magnetic radiator assembly generates magnetic fields at the subject's head, thereby defining a second FOR. A processor: processes field sensor signals to acquire location coordinates of the sensors in the second FOR; segments a tomographic image of the subject, having a third FOR, to identify the subject's face in the third FOR; computes a first transformation between the third and first FORs to map the tomographic face image to the 3D optical image; maps the optical landmarks to the third FOR; maps the respective location coordinates of the sensors to the first FOR; and computes a second transformation between the second FOR and the third FOR.

U.S. Pat. No. 11,481,909 Describes a method and apparatus for performing facial registration includes selecting a plurality of target locations for registration. It is indicated that registration is taking place at each of the plurality of target locations, and it is indicated that registration is complete at the each of the plurality of target locations as each target location is registered. Once the registration of all target locations is complete, it is indicated to the system.

U.S. Pat. No. 10,136,836 Describes an apparatus, including a ferromagnetic sheet and at least one radiator, mounted in proximity to the ferromagnetic sheet and configured to radiate a magnetic field into a region in proximity thereto. The apparatus further includes a solid sheet of thermal insulation, mounted between the ferromagnetic sheet and the at least one radiator so as to prevent transfer of thermal energy from the at least one radiator to the ferromagnetic sheet.

### GENERAL DESCRIPTION

Patient tracker (PT) is a sensor that may be attached to a patient and provide physicians or other medical units with information on patient's location and general movements. PTs may be used during various medical procedures to align medical equipment and operation to patient's movement. It is a generally common situation, where a PT is pulled unintentionally during the procedure, or moves with respect to the patient's body. Such movement of the PT with respect to the patient leads to inaccurate data on patient's location and movements. As a result, additional reregistration process is needed, to determine location of the PT with respect to the patient.

The present disclosure provides a method, and corresponding system, enabling detection between movement of a PT with the patient and movement of the PT with respect to the patient. More specifically, a system operating to determine location and/or orientation of the patient's head (or other body organs) in accordance with PT location. In response to input data indicating movement of the PT, the system of the present disclosure operates to determine if the identified movement relates to (i) movement of the patient's head (or other organs) with the PT, or (ii) movement of the PT with respect to the patient. If this processing determines that the PT movement relates to movement of the user's tracked organ, the respective movement is used to identify user's organ location. Alternatively, if the processing determined that the detected movement relates to PT movement with respect to the patient's body, the system may generate a request for repeated registration in order to align PT location to the patient's organ.

The technique of the present disclosure is based on the inventors' understanding that movement of a point element located on a rigid body typically results in certain correlation between translation movement and rotation movement. Accordingly, the technique of the present disclosure utilizes location data transmitted by one or more patient trackers (PTs) placed on a body of a patient (e.g., on patient's forehead) and processing the location data to differentiate between variation in location associated with translation movement (such as movement along x, y, or z axes) and variation in location associated with rotation movement about one or more axes. The processing further determines a correlation between the translation movement and the rotation movement and compares the determined correlation to prestored threshold data. If the correlation exceeds the threshold, the detected movement is determined as PT movement with the patient's body. If the correlation level is determined to be below the threshold, the detected movement is determined as PT movement with respect to the patient's body. Following classification of the detected movement, a corresponding signal is transmitted.

The corresponding signal may indicate PT movement if correlation exceeds the threshold, allowing an operator (e.g., physician or surgical system) to align operation to patient's body. Alternatively, if the correlation is below the threshold, the output signal may comprise a request for re-registration of PT to the patient's body.

The present technique may be implements by a system comprising at least one processor and memory circuitry (PMC). The processor may be any type of processor capable of executing selected computer readable code in accordance with input data. The PMC may generally be associated with I/O interface for receiving input data, e.g., location data from the one or more PTs, and to generate output data. The output data may be used by the PMC for further processing, e.g., indicating patient's movement to align surgical tools, or be transmitted via a respective user interface such as for requesting repeated registration of PT to patient's body.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 generally illustrates a patient tracker unit and tracking system according to some embodiments of the present disclosure;

FIG. 2 exemplifies a patient tracker location system according to some embodiments of the present disclosure;

FIGS. 3A and 3B exemplify translation and rotation movement detected due to patient's head movement (FIG. 3A) and PT movement (FIG. 3B);

FIGS. 4A, 4B, 4C and 4D are examples graphs showing head movement (derivatives of sampled head location), differences between head movement, absolute difference between head movement and maximum value of head movement respectively, all according to some embodiments of the present disclosure;

FIGS. 4E, 4F, 4G and 4H are examples graphs showing PT movement (derivatives of sampled PT location), differences between PT movement, absolute difference between PT movement and maximum value of PT movement respectively, all according to some embodiments of the present disclosure;”

FIG. 5 exemplify a number of test cases differentiating between PT movement with patient's body or with respect to patient's body according to some embodiments of the present disclosure; and

FIG. 6 illustrates a method of processing location data PT movement with patient's body or with respect to patient's body according to some embodiments of the present disclosure in a way of a block diagram.

### DETAILED DESCRIPTION OF EMBODIMENTS

Patient tracking units, also referred to as patient trackers or PTs, are sensor units typically used to track the location and movement of patients in a healthcare setting. PTs may provide for improving patient safety and efficiency of healthcare procedures and may also be used for collecting movement data relating to patient's move patterns. The use of PTs for tracking patient's movements may often be used in various surgical or other healthcare procedures, e.g., providing surgical tool and surgeons with accurate information of the location of patient's body parts. In other situations, data on patient's movement may be used for analyzing health condition and recovery status.

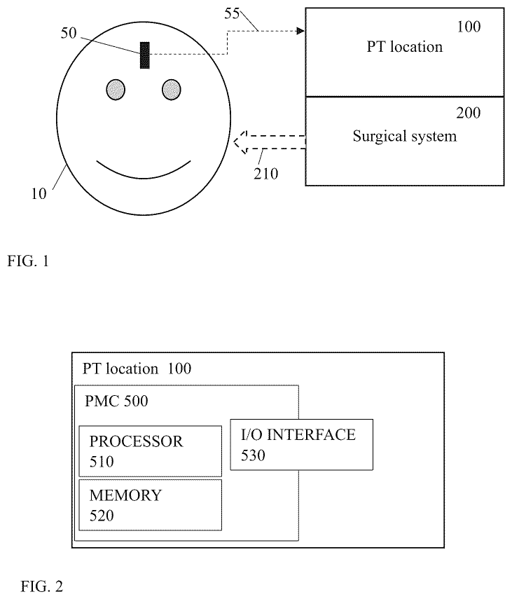

Reference is made to FIG. 1, exemplifying a use of PT in healthcare scenario. FIG. 1 illustrates a patient's head 10, and a PT 50 positioned on the patient's forehead. PT 50 is configured to transmit location data 55 to a PT location system 100 for monitoring patient's location over time. Typically, PT 50 may include one or more location sensors such as one or more accelerometers, magnetic location sensors, gyroscopes, or any other type of location sensor. For example, in some embodiments of the present disclosure, PT 50 may include one or more, e.g., three or more, magnetic coils configured to provide output data indicative of variation in magnetic field. In this example, the respective procedure may be performed in environment having selected magnetic field, allowing the one or more magnetic coils of the PT 50 to detect variation in magnetic field based on location changes of the patient.

The PT location system 100 may be a part, or connected to, a surgical system 200 configured to operate surgical tools in accordance with physician operations, and/or provide a physician with location data indicative of an ongoing healthcare procedure. In certain healthcare procedures, accurate location data of the patient's body part may be important for proper operation. For example, in certain brain surgical procedures, accurate location data of the patient's head may be crucial for successful operation, as the physician needs to be accurate in identifying the surgical site and follow its location.

In other examples, the use of PT can help in preventing patient's from wandering away from designated area, identify patient's location and orientation for patients at risk of falling, and collect patient movement data.

A typical issue associated with the sue of PTs in healthcare environment may be associated with the risk of PT movement from its selected location on the patient's body. Movement of the PT with respect to the patient's body may result in errors in identifying patient's location. While in some scenarios this may result in small inaccuracies, certain health relates procedures, such as surgical procedures, may require accurate location. In such surgical procedure, small errors in patient location data may result in misalignment of tools and cause damage to the patient. The present disclosure provides for a method and system for identifying if received location data indicates movement of the PT with the patient (proper move) or if the PT is moved with respect to the patient (improper move) and provide corresponding output to a physician or to the surgical system 200. The present technique utilizes processing of the location data 55, to identify a correlation level between translation movement and rotation movement of the PT to differentiate between patient's movement with the PT over PT movement with respect to the patient.

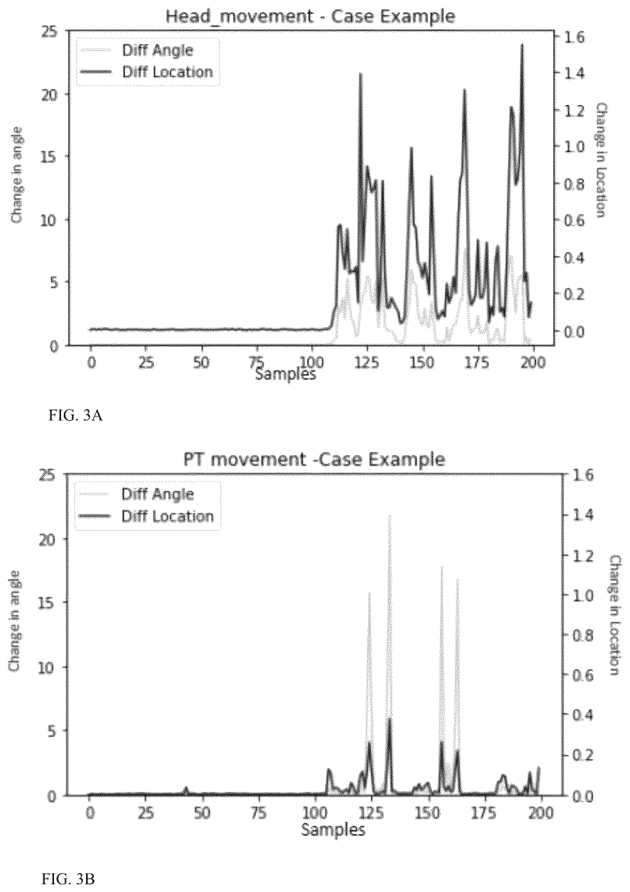

The technique of the present disclosure may be performed by one or more processors and memory circuitries (PMC). FIG. 2 illustrates schematically a PT location system 100 including a PMC 500, which in turn includes at least one processor 510 and memory 520. PMC 500 may include or be associated with an I/O interface 530 for receiving input data and for transmitting output data based on processing output. PMC 500 is operatively connected to I/O interface 530 and is configured to provide processing necessary for operating the system as described further below. PMC 500 is configured to execute one or more functional modules in accordance with computer readable instructions implemented on a computer readable memory (e.g., non-transitory memory).

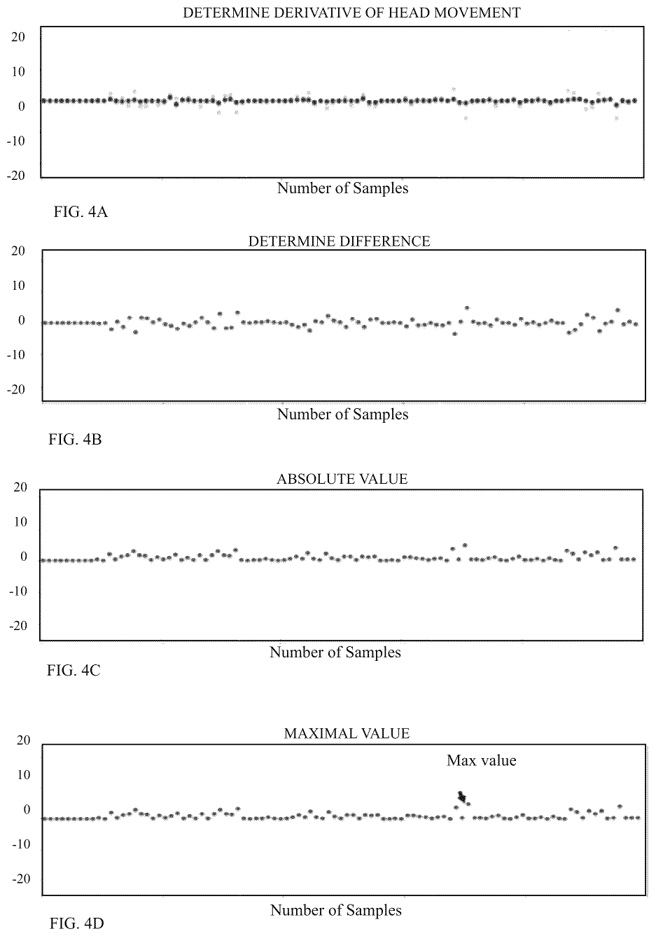

The inventors of the present disclosure have identified that placement of the PT 50 on a patient's head 10 or other body parts, results in certain movement pattern that can be identified. More specifically, the patient's body parts are considered as rigid elements having certain rotation axes associated with connecting joints. As a result, PT movement with the patient's body is characterized by certain correlation between rotational movement and translation movement. This is while movement of the PT by itself, being disconnected to the patient's body parts, does not show such correlation, as the PT moves as a small object that is not directly connected to any remote axis (joint). This is exemplified in FIGS. 3A and 3B showing measured translation and rotation movement. FIG. 3A shows the movement parameters based on PT location data as a result of head movement, and FIG. 3B shows movement parameters associated with PT movement with respect to the patients' body. #

More specifically, FIG. 3A shows derivatives of the location data obtained from a PT when the movement is associated with patient's head's movement and the PT is located on the patient's forehead. The graph shows location derivative in bold curve and rotation (angular) derivative in gray curve. FIG. 3B shows similar measurements associated with a situation where the PT moves without head movement of the patient, i.e., location of the PT on the patient's head changes. As shown in FIGS. 3A and 3B, head movement of the patient is characterized by correlation between the translation and rotation movements. This is while movement of the PT itself, without head movement of the patient, shows low correlation between translation movement and rotation movement.

In accordance with the data exemplified in FIGS. 3A and 3B, the present disclosure utilizes providing PT location data and processing of the PT location data to determine variation associated with translation movement. For example, in some embodiments, the translation variation may be determined by linear derivative operators [d/dx, d/dy, d/dx]. To simplify processing, the absolute value may be used, providing a scalar output for translation location variation. Further, the present disclosure utilizes determining a rotation location variation. This may be determined in some embodiments in accordance with a relation between the magnitude of change in location (Δr=sqrt [(xₜ₁−xₜ₀)²+ (yₜ₁−yₜ₀)²+(zₜ₁−zₜ₀)²] divided by magnitude of location vector (∥rₜ₁∥=sqrt [xₜ₁²+yₜ₁²+zₜ₁²].

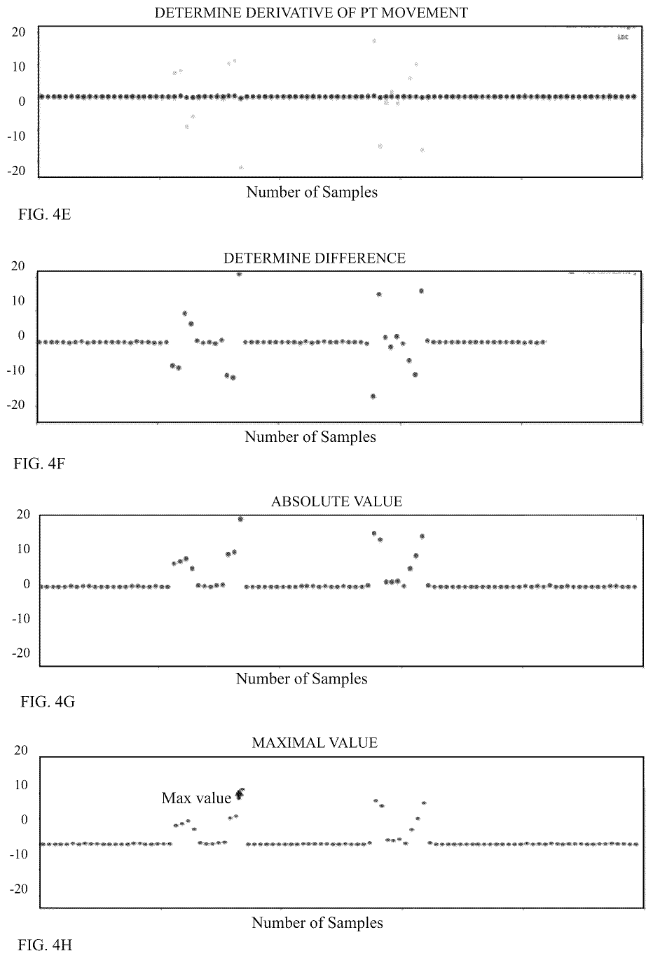

FIGS. 4A to 4H exemplifies processing operations and exemplary measurements and results associated with head movement (FIGS. 4A to 4D) and PT movement (FIGS. 4E to 4H) with respect to the patient's head. As shown, in response to providing PT location data, the processing may operate to determine derivative of the location data, including translation location variation and rotation location variation. To determine a correlation level between the translation movement and rotation movement, the processing in this specific example operates to determine difference between the translation derivation and rotation derivation, and further determined absolute value of the difference. The example of FIGS. 4D and 4H further illustrates determining a maximal value of the difference, for determining a dynamic threshold differentiating between PT movement with respect to the patient's body and PT movement with patient's body. As shown, the maximal value determined for head movement situation (FIG. 4D), is lower with respect to the maximal value determine for PT movement (FIG. 4H) with respect to the head in this example. This illustrates low correlation between translation movement and rotation movement for the case of PT movement, while head movement with the PT shows higher correlation between the translation and rotation movement.

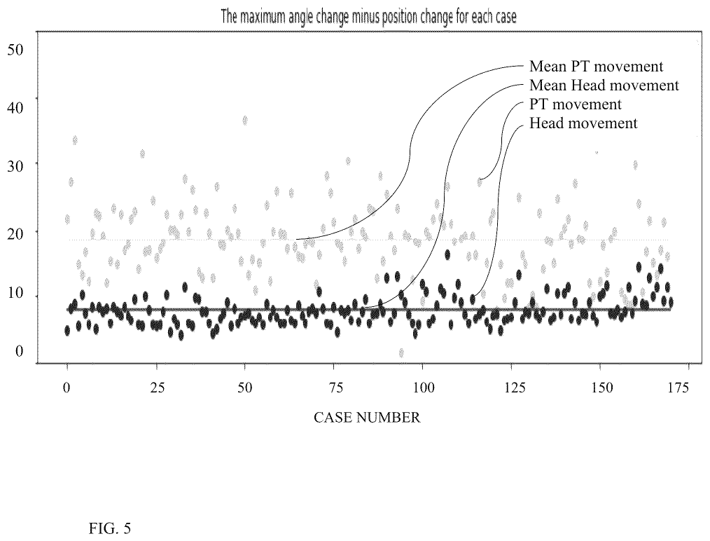

Such maximal values, determined for a set of about 160 test data sets is illustrated in FIG. 5. As shown, the mean maximal difference value determined for PT movement with respect to patient's head measured at 18.5 [AU], where mean maximal difference for head movement is measured as 7.9 [AU]. Additionally, standard deviation of the maximal value for PT movement is measured at 5.57, and standard deviation of the maximal value for head movement is measured at 2.16.

Based on the above results, differentiating between PT movement with respect to patient's body and body (e.g., head) movement with the PT, can be done utilizing correlation between translation and rotation movement components. Such correlation can be determined by maximal difference value, time correlation, or any other suitable correlation operation. The PT location system 100 as illustrated in FIG. 1 may operate to determine correlation between the translation and rotation movement components and based on the correlation, identify if the movement relates to patient's body movement, which is used for directing surgical tools and physician operation, or if the movement relates to PT movement with respect to the patient's body. In the latter case, the PT location system may generate an output signal requesting that the PT location is re-aligned to the patient's body, to support further surgical (or other healthcare related) operations.

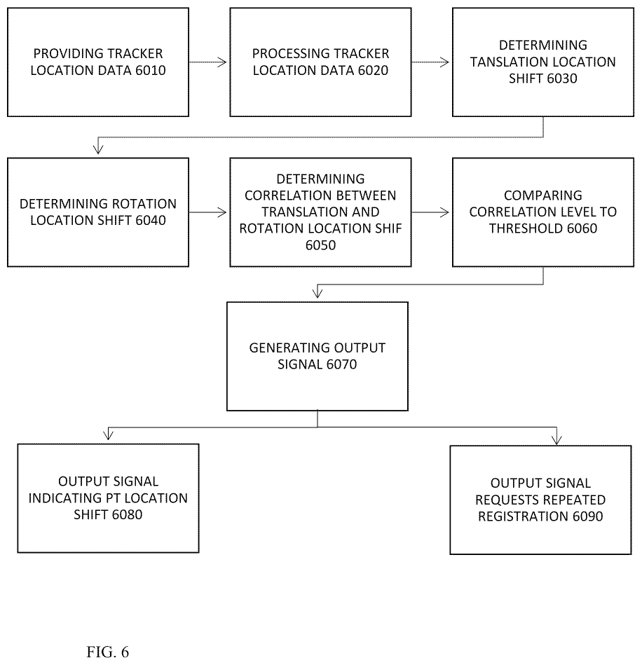

In this connection, reference is made to FIG. 6 illustrating in a way of a block diagram, a method of processing location data PT movement with patient's body or with respect to patient's body according to some embodiments of the present disclosure. As shown in FIG. 6, the present disclosure provides a method including providing (or obtaining) PT location data 6010, typically received as a series of location signals transmitted by the PT at a selected sampling rate and processing the PT location data 6020. Processing of the PT location data may include in some embodiments various actions such as storing PT location data and identifying PT identity. Additionally, to determine PT movement data, the processing may include determining translation location shift (movement) 6040 and determining rotation location shift (movement) 6050. In some embodiments, translation movement may be determined by determining difference between location data at time t and location data at time t+Δt along one or more axes, and in some further embodiments, determining magnitude of the difference. Further, in some embodiments, rotation movement may be determined based on a direction of the derivative and determining a rotation matrix accordingly. For example, the rotation matrix may be determined based on cross product between the derivative vector and each of three axes.

Upon determining rotation and translation location shifts, the method further includes determining a correlation between the translation and rotation location shifts 6050. In some embodiments, determining correlation may include processing of a selected set of movement samples, e.g., collection of PT location data at a sampling rate of 100 Hz, and determining correlation between a set of previous 50 data pieces. As described above high correlation between the translation and rotation location shifts indicate movement of the PT with a respective patient's body part. This is while low correlation indicates PT movement that is not correlated with patient's respective body part. The correlation level may be compared to a pre-store or predetermined threshold 6060, to determine movement type based on the PT location data. In accordance with determining movement type of the PT, the method may include generating an output signal indicating the type of movement 6070.

The output signal may include data indicative of the PT location data and an indication that the PV movement is associated with movement of the respective patient's body part. Alternatively, the output signal may include an indication that the PT movement is likely no associated with movement of the respective patient's body part, and repeated registration of the PT location may be needed.

The present technique may thus provide a method and respective system enabling classification of PT location data to differentiate between PT movement associated with a respective patient' body part and PT movement with respect to the patient's body parts (i.e., PT moves from its location on the patient's body). In accordance with sampling rate, the present technique may enable high sensitivity, and can identify type of movement for location shifts of 1 mm or below.

### EXAMPLES

Example 1: A method comprising:
(a) providing location data (55) from one or more patient trackers (PT) (50) placed on a patient's body (10);
(b) processing said location data (6020) and identifying translation data (6030) indicative of translation movement of the PT and rotation data (6040) indicative of angular orientation variation of the PT;
(c) determining a correlation (6050) between said translation data and said rotation data;
(d) generating (6070) output signal (i) indicative of PT movement if correlation exceeds a selected threshold, or (ii) comprising an alert on PT movement with respect to patient if correlation is below said selected threshold.

Example 2: The method of example 1, wherein said output signal comprising an alert on PT (50) movement further comprises an indication (6090) on required registration of said PT to the patient's body.

Example 3: The method of example 1 or example 2, wherein said determining correlation (6050) between said translation data and said rotation data comprises determining absolute value of said translation data and of said rotation data and determining correlation between absolute values of said translation data and said rotation data.

Example 4: The method of any one of examples 1-3, providing for differentiating between PT (50) movement with patient and PT (50) movement with respect to patient in accordance with movement below 1 millimeter.

Example 5: The method of any one of examples 1-4, wherein said one or more PT (50) comprises at least one head mounted PT (50).

Example 6: The method of any one of examples 1-5, operable with a single PT (50) mounted on patient's body and providing location data in three axes.

Example 7: A system (100) comprising at least one processing and memory circuitry (PMC) (500), and a respective input/output (I/O) interface (530); said at least one PMC (500) is adapted for receiving and processing location data (55) collected by one or more patient (10) trackers (PT) (50) by determining correlation between rotation data and translation data of said PT (50), and for determining, in response to detected movement if said PT (50) is moving with said patient or with respect to said patient (10) in accordance with said correlation.

Example 8: The system of example 7, wherein said PMC (500) is further configured for generating an output signal indicating of (i) PT movement if correlation exceeds a selected threshold (6080), or (ii) an alert on PT movement with respect to patient if correlation is below said selected threshold (6090).

Example 9: The system of example 7 or example 8, wherein said PMC (500) is configured for generating an output signal indicating a need for registration of said PT (50) to the patient's body (10) in response to determining that said correlation is below a selected threshold.

Example 10: The system of any one of examples 7-9, wherein said PMC (500) is adapted to receive location data (55) from one or more PT (50), and to determining rotation data indicative of angular variation of location, and translation data indicative of translation movement of the PT (50).

Example 11: The system of any one of examples 7-10, wherein said determining correlation between said translation data and said rotation data comprises determining absolute value of said translation data and of said rotation data and determining correlation between absolute values of said translation data and said rotation data.

Example 12: The system of any one of examples 7-11, operable for differentiating between PT (50) movement with patient and PT (50) movement with respect to patient in accordance with movement below 1 millimeter.

Example 13: The system of any one of examples 7-12, operable with a single PT (50) mounted on patient's body (10) and providing location data in three axes.

Example 14: The system of any one of examples 7-13, further comprising one or more PTs (50) comprising a sensor arrangement and transmitter circuitry and configured for transmitting location data determining by said sensor arrangement in a selected sampling rate.

Example 15: The system of example 14, wherein said one or more PTs (50) comprises at least one head mounted PT (50).

Example 16: The system of example 14 or example 15, wherein said one or more PTs (50) comprise at least one PT (50) comprising a magnetic sensor arrangement.

Example 17: The system of any one of examples 7-16, adapted for differentiating between PT movement with patient and PT movement with respect to patient in accordance with location data collected from a single PT.

Example 18: A program storage device readable by machine, tangibly embodying a program of instructions executable by the machine to perform a method comprising:
(a) being responsive to receive location data from one or more patient trackers (PT) placed on a patient's body;
(b) processing said location data and identifying translation data indicative of translation movement of the PT and rotation data indicative of angular orientation variation of the PT;
(c) determining a correlation between said translation data and said rotation data;
(d) generating output signal (i) indicative of PT movement if correlation exceeds a selected threshold, or (ii) comprising an alert on PT movement with respect to patient if correlation is below said selected threshold.

Example 19: The program storage device of claim 18, wherein said output signal comprising an alert on PT movement further comprises an indication on required registration of said PT to the patient's body.

Example 20: The program storage device of example 18 or example 19, wherein said determining correlation between said translation data and said rotation data comprises determining absolute value of said translation data and of said rotation data and determining correlation between absolute values of said translation data and said rotation data.

1. A method comprising:
(a) providing location data from one or more patient trackers (PT) placed on a patient's body;
(b) processing said location data and identifying translation data indicative of translation movement of the PT and rotation data indicative of angular orientation variation of the PT;
(c) determining a correlation between said translation data and said rotation data;
(d) generating output signal (i) indicative of PT movement if correlation exceeds a selected threshold, or (ii) comprising an alert on PT movement with respect to patient if correlation is below said selected threshold.

2. The method of claim 1, wherein said output signal comprising an alert on PT movement further comprises an indication on required registration of said PT to the patient's body.

3. The method of claim 1, wherein said determining correlation between said translation data and said rotation data comprises determining absolute value of said translation data and of said rotation data and determining correlation between absolute values of said translation data and said rotation data.

4. The method of claim 1, providing for differentiating between PT movement with patient and PT movement with respect to patient in accordance with movement below 1 millimeter.

5. The method of claim 1, wherein said one or more PT comprises at least one head mounted PT.

6. The method of claim 1, operable with a single PT mounted on patient's body and providing location data in three axes.

7. A system comprising at least one processing and memory circuitry (PMC) (500), and a respective input/output (I/O) interface; said at least one PMC is adapted for receiving and processing location data collected by one or more patient trackers (PT) by determining correlation between rotation data and translation data of said PT, and for determining, in response to detected movement if said PT is moving with said patient or with respect to said patient in accordance with said correlation.

8. The system of claim 7, wherein said PMC is further configured for generating an output signal indicating of (i) PT movement if correlation exceeds a selected threshold, or (ii) an alert on PT movement with respect to patient if correlation is below said selected threshold.

9. The system of claim 8, wherein said PMC is configured for generating an output signal indicating a need for registration of said PT to the patient's body in response to determining that said correlation is below a selected threshold.

10. The system of claim 7, wherein said PMC is adapted to receive location data from one or more PT, and to determining rotation data indicative of angular variation of location, and translation data indicative of translation movement of the PT.

11. The system of claim 7, wherein said determining correlation between said translation data and said rotation data comprises determining absolute value of said translation data and of said rotation data and determining correlation between absolute values of said translation data and said rotation data.

12. The system of claim 7, operable for differentiating between PT movement with patient and PT movement with respect to patient in accordance with movement below 1 millimeter.

13. The system of claim 7, operable with a single PT mounted on patient's body (10) and providing location data in three axes.

14. The system of claim 7, further comprising one or more PTs comprising a sensor arrangement and transmitter circuitry and configured for transmitting location data determining by said sensor arrangement in a selected sampling rate.

15. The system of claim 14, wherein said one or more PTs comprises at least one head mounted PT.

16. The system of claim 14, wherein said one or more PTs comprise at least one PT comprising a magnetic sensor arrangement.

17. The system of claim 7, adapted for differentiating between PT movement with patient and PT movement with respect to patient in accordance with location data collected from a single PT.

18. A program storage device readable by machine, tangibly embodying a program of instructions executable by the machine to perform a method comprising:
(a) being responsive to receive location data from one or more patient trackers (PT) placed on a patient's body;
(b) processing said location data and identifying translation data indicative of translation movement of the PT and rotation data indicative of angular orientation variation of the PT;
(c) determining a correlation between said translation data and said rotation data;
(d) generating output signal (i) indicative of PT movement if correlation exceeds a selected threshold, or (ii) comprising an alert on PT movement with respect to patient if correlation is below said selected threshold.

19. The program storage device of claim 18, wherein said output signal comprising an alert on PT movement further comprises an indication on required registration of said PT to the patient's body.

20. The program storage device of claim 18, wherein said determining correlation between said translation data and said rotation data comprises determining absolute value of said translation data and of said rotation data and determining correlation between absolute values of said translation data and said rotation data.
